SEQUENCE LISTING (30 sequences)

SEQ ID NO: 1 - DNA, 27 bp, Artificial
  Human
cagcagcagcagcagcagcagcagcag

SEQ ID NO: 2 - DNA, 6 bp, Artificial
  Human
gtcgtc

SEQ ID NO: 3 - DNA, 12 bp, Artificial
  Human
gtcgtcgtcgtc

SEQ ID NO: 4 - DNA, 15 bp, Artificial
  Human
gtcgtcgtcgtcgtc

SEQ ID NO: 5 - DNA, 21 bp, Artificial
  Human
gtcgtcgtcgtcgtcgtcgtc

SEQ ID NO: 6 - DNA, 27 bp, Artificial
  Human
cggcggcggcggcggcggcggcggcgg

SEQ ID NO: 7 - DNA, 6 bp, Artificial
  Human
gccgcc

SEQ ID NO: 8 - DNA, 12 bp, Artificial
  Human
gccgccgccgcc

SEQ ID NO: 9 - DNA, 15 bp, Artificial
  Human
gccgccgccgccgcc

SEQ ID NO: 10 - DNA, 21 bp, Artificial
  Human
gccgccgccgccgccgccgcc

SEQ ID NO: 11 - DNA, 20 bp, Artificial
  Human
atggtgcacctgactcctga

SEQ ID NO: 12 - DNA, 21 bp, Artificial
  Human
ggagaagtctgccgttactgc

SEQ ID NO: 13 - DNA, 41 bp, Artificial
  Human
gcagtaacggcagacttctcctcaggagtcaggtgcaccat

SEQ ID NO: 14 - DNA, 41 bp, Artificial
  Human
atggtgcacctgactcctgaggagaagtctgccgttactgc

SEQ ID NO: 15 - DNA, 15 bp, Artificial
  Human
acggcagacttctcc

SEQ ID NO: 16 - DNA, 36 bp, Artificial
  Human
cggcggcggcggcggcggcggcggcggcggcggcgg

SEQ ID NO: 17 - DNA, 30 bp, Artificial
  Human
ctgctgctgctgctgctgctgctgctgctg

SEQ ID NO: 18 - DNA, 60 bp, Artificial
  Human
ctgctgctgctgctgctgctgctgctgctgctgctgctgctgctgctgctgctgctgctg

SEQ ID NO: 19 - DNA, 9 bp, Artificial
  Human
cagcagcag

SEQ ID NO: 20 - DNA, 59 bp, Streptococcus salivarius
  misc_feature (34)..(36): n is a, c, g, or t
taggtgaattaataagcctagggacttgattttnnncaagttacggcgagtgaactggc

SEQ ID NO: 21 - DNA, 59 bp, Streptococcus vestibularis
  misc_feature (34)..(36): n is a, c, g, or t
taggtgaatcaataagcctagggacttgattttnnncaagttacggcgagcgaactagc

SEQ ID NO: 22 - DNA, 59 bp, Streptococcus orisratti
taggcgaaaaaataagcctaggggggtagtcttttctgccctacggcgagtaaaatggc

SEQ ID NO: 23 - DNA, 59 bp, Streptococcus canis
  misc_feature (29)..(30): n is a, c, g, or t
  misc_feature (35)..(36): n is a, c, g, or t
taggcgaacaaataagcctagggatgtgnncttgnncacattacggcggagaaaatggc

SEQ ID NO: 24 - DNA, 59 bp, Streptococcus equi zooepid
  misc_feature (29)..(30): n is a, c, g, or t
  misc_feature (36)..(36): n is a, c, g, or t
taggcgaacaaataagcctagggatgtgnntttgancacattacggcgagtgaaaaggc

SEQ ID NO: 25 - DNA, 59 bp, Streptococcus dysgal equi
  misc_feature (29)..(30): n is a, c, g, or t
  misc_feature (35)..(36): n is a, c, g, or t
taggcgaacaaataagcctagggatgtgnncttanntacattacggcgaagaaaatggc

SEQ ID NO: 26 - DNA, 59 bp, Streptococcus parauberis
  misc_feature (28)..(31): n is a, c, g, or t
taggcgaaaaaataagcctagggatgcnnnnagaaatgcattacggcgaaagaacgagc

SEQ ID NO: 27 - DNA, 59 bp, Streptococcus iniar
  misc_feature (29)..(30): n is a, c, g, or t
taggcgaaaaaataagcctaggaatgtannctttagtacattacggcgagtgaaatggc

SEQ ID NO: 28 - DNA, 59 bp, Streptococcus pyogenes
  misc_feature (29)..(30): n is a, c, g, or t
  misc_feature (35)..(36): n is a, c, g, or t
taggcgaacacataagcctagggatgtgnncatanncacattacggcgaaggaaatggc

SEQ ID NO: 29 - DNA, 59 bp, Streptococcus phocae
  misc_feature (29)..(30): n is a, c, g, or t
  misc_feature (35)..(36): n is a, c, g, or t
taggcgaaaaaataagcctagggatgtgnnattgnncacattacggcgaaagaactggc

SEQ ID NO: 30 - DNA, 59 bp, Streptococcus pluranimalium
  misc_feature (33)..(33): n is a, c, g, or t
  misc_feature (36)..(36): n is a, c, g, or t
taggcgaaaaaataagcctagggacgtatgatngantacgttacggcaggtaaaatggc

(19) United States
(12) Patent Application Publication
(10) Pub. No.: US 2008/0076118 A1
(43) Pub. Date: Mar. 27, 2008

(54) Oligonucleotide Ligation Assay By Detecting Released Pyrophosphate

(76) Inventors: Nigel Tooke, Knivsta (SE); Bjorn Ekstrom, Uppsala (SE)

Correspondence Address: ALBIHNS STOCKHOLM AB, BOX 5581, LINNEGATAN 2, SE-114 85 STOCKHOLM; SWEDENn, STOCKHOLM, SE

(21) Appl. No.: 10/562,134

(22) PCT Filed: Jun. 30, 2004
(86) PCT No.: PCT/EP04/07090

§ 371 Date: Mar. 16, 2007

Related U.S. Application Data
(60) Provisional application No. 60/481,043, filed on Jun. 30, 2003.
(60) Provisional application No. 60/481,319, filed on Sep. 1, 2003.

(30) Foreign Application Priority Data
Jun. 30, 2003 (SE) 0301951-0

(51) Int. Cl. C12Q 1/68

(52) U.S. Cl. 435/6

(57) ABSTRACT

The present invention is related to a new method for determining the presence of genetic element(s), such as nucleotide repeat(s), or marker(s) for microbial typing, in a nucleic acid sample. The method is based on performing a ligation reaction where after a by-product of the ligation is detected and used to determine the number of nucleotidc repeat units in a nucleic acid sample possibly comprising a nucleotide repeat. The invention is also related to kits for performing the method of the present invention and compositions comprising components for performing the present invention.

### TECHNICAL FIELD

The present invention relates to a method for determining the presence of genetic element(s), such as nucleotide repeat(s), or marker(s) for microbial typing, in a nucleic acid sample by performing a ligation reaction and detecting a ligation by-product.

### BACKGROUND OF THE INVENTION

Genetic variation is often linked to disease and identification of genetic variation is an important tool in clinical diagnosis. Genetic variation involve differences at the level of single bases (mutations and SNPs involving base changes, insertions, or deletions), several bases (typically involving codon deletions), or varying numbers of repeated sequences of varying lengths, up to multiplication of whole genes.

Expansion of nucleotide repeats have been shown to be linked to different genetic diseases. Examples include Fragile X (Webb, T P et al, 1986, Am. J. Med. Genet. 23: 573, Gustavson, K H et al, 1986, Am. J. Med. Genet. 23: 581), myotonic dystrophy (Harper, P S et al, 1989, Myotonic Dystrophy, 2d Ed., London, England, WB Saunders Co, 1989) and Huntington's disease (MacDonald, M E et al, 1993, Cell 72:971). These nucleotide repeats have been analysed using, for example, PCR-based assays and Southern blotting techniques. Furthermore, di-, tri-, tetra-, penta- and hexa-nucleotide repeats have been extensively used in gene mapping projects and in different forensic applications, like paternity tests.

Pyrosequencing™ is a sequencing-by-synthesis method developed at the Royal Institute of Technology in Stockholm, based on the detection of the release of pyrophosphate and enzymatic nucleotide degradation, (U.S. Pat. No. B1-6,210,891 and U.S. Pat. No. B1-6,258,568). In this sequencing-by-synthesis-method, in contrast to conventional Sanger sequencing, the nucleotides are added one by one during the sequencing reaction. The enzyme mix used consists of four different enzymes; DNA polymerase, ATP-sulfurylase, luciferase and apyrase. The nucleotides are sequentially added according to a specified order dependent on the template and determined by the user. If the added nucleotide matches the template, the DNA polymerase will incorporate it into the growing DNA strand and pyrophosphate, PPi, will be released. The ATP-sulfurylase converts the PPi into ATP, and the third enzyme, luciferase, transforms the ATP into a light signal. Following these reactions, the fourth enzyme, apyrase, degrades the excess nucleotides and ATP, and the template will at that point be ready for the next reaction cycle, i.e. another nucleotide addition. Since no PPi is released unless a nucleotide is incorporated, a light signal will be produced only when the correct nucleotide is incorporated. Pyrosequencing™ is a real time DNA sequencing method based on sequencing-by-synthesis. The method has proven to be a fast and accurate method for SNP (single nucleotide polymorphism) scoring, sequencing of shorter DNA stretches (signature tags), and assessment of allele frequencies (Ronaghi, M., Uhlen, M., Nyrén, P., A sequencing method based on real-time pyrophosphate (1998), Science 281, 363-365.; Alderborn, A., Kristofferson, A., Hammerling, U. Determination of single nucleotide polymorphisms by real-time pyrophosphate DNA sequencing (2000), Genome Res. 10:1249-1258).

Sequencing-by-synthesis, in the form of Pyrosequencing™, has many advantages in the analysis of genetic variation. For example, compared to other techniques used for SNP analysis, such as hybridisation techniques, minisequencing, RFLP and SSCP, sequencing-by-synthesis confirms that the correct SNP position is examined, by presenting the surrounding sequence and not only the polymorphic positions. Furthermore, Pyrosequencing™ is a rapid technique that provides real time analysis without separation steps and in an automatable fashion, which is a benefit compared to SSCP and RFLP and it is therefore an attractive method for genetic analysis.

However, Pyrosequencing™ is most effective when analysing short stretches of nucleotides in the range 1-100 bases. The method is limited primarily by the accumulation of the products of out-of-phase primer extension, so-called ‘shift’. Shift is the result of incomplete or excessive extension of the primer due to, for example, the presence of sub-optimal levels of nucleotides. The homogeneous Pyrosequencing™ reaction involves a competition between the DNA polymerase that incorporates the correct nucleotide, and apyrase that degrades unincorporated nucleotides. If the level of correct nucleotide drops below the optimum for the DNA polymerase during the incorporation step then further extension of some primers will be incomplete. The result is a population of extended primer molecules that are one or more bases shorter than the correct, fully extended primers (so-called minus shift). On the other hand, if apyrase is not sufficiently effective in degrading nucleotides then excess, unincorporated nucleotides can remain in the reaction and be incorporated in certain situations when the next, correct nucleotide is presented and incorporated and the sequence permits further extension using the undesired nucleotide background (so-called plus shift). An additional phenomenon involves templates with complex secondary structures that disturb the activity of the DNA polymerase, and thus cause incomplete incorporation and minus shift. These errors naturally increase with increasing number of primer extensions. Therefore, the accuracy of techniques that are dependent on elongation of nucleic acids by single nucleotide extension for estimating the number of nucleotide repeats decreases with increasing number and length of nucleotide repeats.

U.S. Pat. No. 6,309,829 describes a method for analysing the number of nucleotide repeats in which a primer is annealed to a template nucleic acid and extended one nucleotide at a time, in which the nucleotide is labelled and gives a measurable signal as an indication that the extension has taken place. The label of the last added nucleotide is then removed before the next nucleotide is added. The extension is continued until the signal in one cycle is substantially less than the signal in the foregoing reaction.

Methods based on ligation reactions have recently become an important tool in the analysis of genetic variation. U.S. Pat. No. 5,695,933 describes a method, called RED (repetitive expansion detection) for detecting the expansion of nucleotide repeats in which oligonucleotides complementary to the repetitive sequences are annealed to the repetitive sequences in the presence of a ligase. The ligase then joins juxtaposed oligonucleotides to produce multimers of ligated oligonucleotides. The complex of multimers and target nucleotide is then denatured and a new round of annealing, ligation and denaturation is started. The length of the multimers is finally determined by techniques involving gel separation. The length of the multimers produced indicates the length of the expanded oligonucleotide repeat, which in turn can indicate the genetic condition of a sampled individual. This method allows the number of repeats to be estimated, even when longer stretches of nucleic acids are to be analysed. However, this analytical method involves gel-based separation followed detection by hybridisation, or direct detection, of radio-labelled DNA. This detection is very time-consuming and laborious and does not allow real-time analysis. Furthermore, gel based separation have low resolution of larger DNA fragments, which will render in unclear results regarding number of repeats.

In summary, there is still a need for new methods allowing a rapid and accurate analysis of genetic variation, including the number of nucleotide repeats in a nucleic acid sample.

### SUMMARY OF THE INVENTION

The present inventors have surprisingly found a new method of not only estimating the number of nucleotide repeats, but also of determining the presence of other genetic elements, such as markers for microbial typing, by performing a ligation reaction and detecting a ligation by-product. In a first embodiment the present invention relates to a method for determining the presence of genetic element(s), such as nucleotide repeat(s), or marker(s) for microbial typing, in a nucleic acid sample, which method comprises the steps of:
a) providing the nucleic acid sample comprising the genetic element(s);
b) providing oligonucleotide(s) that are completely or partially complementary to the region(s) comprising the genetic element(s) of said nucleic acid sample;
c) annealing said oligonucleotide(s) to said nucleic acid sample;
d) ligating said oligonucleotide(s) annealed to said nucleic acid sample to each other using a ligase enzyme; and
e) detecting a ligation-by-product to determine whether a ligation reaction has occurred, as a measure of the presence of the genetic element(s),

wherein steps a)-e) are performed simultaneously or subsequently or in any combination of subsequent steps.

Since the amount of ligation by-product produced is proportional to the number of ligations reactions that have occurred, the number of ligation reactions can be calculated by determining how much ligation by-product is produced. Alternatively, in one embodiment of the present invention ligation cycles are performed subsequently and the occurrence of one ligation reaction is detected before the next is initiated. In this case it is only necessary to detect whether or not a ligation reaction has occurred (and not the actual amount of ligation by-product produced) to determine the number of genetic elements that are present, such as repeat units in a repeated nucleic acid sequence. The present invention also relates to kits to perform the present method and compositions comprising components necessary for performing the present method. Since, in the present invention, a ligation by-product, and not the ligated product in itself, is analysed, the present invention opens up new ways of determining the number of nucleotide repeats. The present invention allows the number of nucleotide repeat units in a nucleotide repeat sequence, or markers for microbial typing, to be faster, in a less laborious way and more accurately determined, even with very long sequences comprising genetic elements, such as nucleotide repeats, than has earlier been possible. Additionally, the analysis can be performed in real-time. The present invention also allows the number of genetic elements, such as nucleotide repeats in a heterozygous sample to be determined, if necessary.

### DRAWINGS

FIG. 1. Description of a so-called “end-point” analysis according to the present invention.

FIG. 2. Description of a so-called “step-wise” analysis according to the present invention.

FIG. 3. Ligation of two oligonucleotides at a variable position in a gene with a thermocycled ligation reaction followed by bioluminescent detection.

FIG. 4. Ligation of two oligonucleotides at a variable position in a gene with the ligation reaction linked to bioluminescent detection. Combination of ligase and PPDK steps on pre-annealed primer/template complex.

FIG. 5. Detection of difference in number of CTG repeats.

FIG. 6. (a) Ligation of two oligonucleotides at a variable position in a gene. Use of a ATP-dependent ligase (T4 DNA ligase) and dATP as a co-factor. Apyrase is used to reduce excess of co-factor; (b) Comparison of ligation of two oligonucleotides at a variable position using two ligation methods (ATP-dependent and NAD⁺-dependent ligase).

FIG. 7. Alignment of sequences from a region of the rnpB gene from a number of species of streptococci.

FIG. 8. Annealing of probes to a region of different species.

### DEFINITIONS

By a “genetic element” is meant a detectable feature of a nucleic acid molecule, such as a nucleotide repeat or a marker for microbial typing.

By a “region comprising the genetic element” is meant a sequence of nucleotides in a nucleic acid molecule. The region may be identical to the genetic element, such as a nucleotide repeat, or it may comprise one or more genetic elements, such as markers for microbial typing. Typically, the region comprising the genetic element is a region that corresponds to an oligonucleotide as used in the methods of the invention.

By a “marker for microbial typing” is meant one or more nucleotide positions in a nucleic acid sample that are characteristic for a specific microbial type, and thus is used in order to type a nucleic acid sample.

By a “ligation pattern” is meant the pattern of ligation events, i.e. the number of oligonucleotides that are ligated and/or the identity of the oligonucleotides. Typically, the ligation pattern of a sample is characteristic for the type of the sample. Consequently, by a “reference pattern” is meant the pattern of ligation events for a known reference sample.

By “nucleotide repeat” is meant a contiguous repetition of a specific sequence of two, three, four up to thousands of nucleotides. “Repeat unit” is one nucleotide repeat sequence. If the repeat unit is between 2 and 6 nucleotides, the nucleotide repeats are called micro-satellites; if they are 7 to approximately 30 nucleotides, they are called mini-satellites; if they are larger then 30 they are termed satellites. The term “nucleotide repeats” also includes the term short tandem repeats and variable number of tandem repeats, VNTR. In some genetic disorders the repeat unit can be repeated several thousands of times. Humans and other organisms with linear chromosomes have at their chromosome ends the telomeres built up of short repeat units, repeated thousands of times.

By “flanking sequence” is meant any nucleotide sequence outside the nucleotide repeat. This sequence is in many situations used for primers in an amplification step.

By “complementary” is meant that the oligonucleotides have enough complementarity (i.e. ability to base-pair) to be able to anneal to a template nucleic acid under the chosen annealing condition. By complementary is therefore not meant that all nucleotides in all positions have to be complementary to the template nucleic acid, only that the oligonucleotide has enough complementarity to anneal to the template nucleic acid under the chosen annealing conditions.

By “ligation by-product” is meant a product that does not constitute a part of the ligated nucleic acids but is released during the ligation reaction.

By “luciferase” is in the present invention meant any enzyme capable of producing light in an enzymatic reaction that is dependent on ATP. Therefore such enzymes from any species, including mutant and recombinant variants thereof are included in the definition “luciferase” according to the present invention. By luciferase-based assay is meant an assay that employs a luciferase enzyme.

A “luminometric assay” is an assay in which the activity of an enzyme, or the level of a compound, is monitored in a light-emitting reaction, whereby the light-emission is the result of an enzymatic reaction (i.e. the result of an activity of a luciferase enzyme).

### DETAILED DESCRIPTION OF THE INVENTION

Some organisms are capable of producing light in exergonic reaction, leading to bioluminescence. The generic name for the enzyme involved in bioluminescence reactions is luciferase. Different variants of this enzyme have been found in various organisms such as bacteria, insects and dinoflagellates.

Firefly luciferase (EC 1.13.12.7) catalyses the oxidation of D-luciferin in the presence of ATP, magnesium and oxygen. The product, oxyluciferin, is generated in an exited state which decays to the ground state with the emission of a photon. Firefly luciferase has been extensively used in molecular and cell biology, in particular for efficient detection and quantification of ATP, and as a reporter enzyme for studies of gene regulation and expression (Gould, S. J., and Subramani, S. (1988) Firefly luciferase as a tool in molecular and cell biology. Anal. Biochem. 175, 5-13). All enzymes and metabolites involved in ATP converting reactions can be analyzed by the firefly luciferase system (Kricka, L. J. (2000) Application of bioluminescence and chemiluminescence in biomedical sciences. Methods Enzymol. 305, 333-345; Kricka, L. J. (1988) Clinical and biochemical applications of luciferase and luciferins. Anal. Biochem. 175, 14-21; Kricka, L. J. (1991) Chemiluminescent and bioluminescence techniques. Clin. Chem. 37, 1472-1481).

Moreover, luciferase systems have been used in various bioluminescence assays. For example, various fusion proteins comprising luciferase and another protein, such as a RNA-binding protein for RNA identification, or a fusion conjugate of firefly luciferase and a biotin acceptor peptide or a single chain antibody for an immunoassay, are known. Eukaryotic luciferase enzymes employ ATP in reactions producing light. Due to the simplicity with which light can be detected, luciferase-based analysis are an attractive analytic tool for many applications.

In a first embodiment, the invention relates to a method for determining the presence of genetic element(s), such as nucleotide repeat(s), or marker(s) for microbial typing, in a nucleic acid sample, which method comprises the steps of:

a) providing the nucleic acid sample comprising the genetic element(s);

b) providing oligonucleotide(s) that are completely or partially complementary to the region(s) comprising the genetic element(s) of said nucleic acid sample;

c) annealing said oligonucleotide(s) to said nucleic acid sample;

d) ligating said oligonucleotide(s) annealed to said nucleic acid sample to each other using a ligase enzyme; and

e) detecting a ligation-by-product to determine whether a ligation reaction has occurred, as a measure of the presence of the genetic element(s),

wherein steps a)-e) are performed simultaneously, i.e. by mixing all components together, or subsequently, i.e. mixing only the components necessary for each step and adding additional components subsequently. Alternatively, some steps may be performed simultaneously and some steps subsequently. This can be preferable for example when the different steps require different conditions in order to be performed, e.g. different buffers, temperatures etc. All components necessary for the reaction except one can also be mixed prior to analysis and the chain of reactions initiated by addition of the last component.

The present invention is related to the detection of ligation by-product instead of the ligated product in itself. This opens up new ways of detecting whether a ligation event has occurred, which allows faster and more sensitive analysis to be performed compared to prior art.

The nucleic acid sample can be any DNA- or RNA-sample of a biological sample, such as from tissue, cells or blood, which is preferably, but not necessarily, isolated by standard means known to the skilled person. Preferably the nucleic acid sample is a nucleic acid sample from a human subject. Preferably the nucleic sample is DNA. Preferably the nucleic sample is amplified, e.g. by PCR.

In the method according to the present invention the nucleic acid sample is mixed with oligonucleotides complementary to the specific repeated sequence to be analysed and allowed to anneal. Prior to the annealing step the nucleic acid has to be denatured. The skilled person knows how to select suitable conditions for denaturing nucleic acids. The conditions for annealing have to be varied depending on the specific oligonucleotide to be annealed. The skilled person knows how to select the stringency during the annealing for different applications. The length and identity of the oligonucleotides has to be adjusted for each specific region comprising genetic element(s), such as a nucleotide repeat, that is to be detected. All oligonucleotide(s) need a phosphate in the 5′-end in order to work in a ligation reaction.

Today, there are at least 14 documented disorders that affect human beings, which have their origin in trinucleotide repeat expansions. They can be grouped in polyglutamine (8 disorders) and non-polyglutamine diseases (6 diseases).

The 8 disorders, one of which is Huntington's Disease, all share the same repeated codon as their cause: CAG. Since CAG codes for an amino acid called glutamine, these eight trinucleotide repeat disorders are collectively known as polyglutamine diseases.

Polyglutamine diseases have much in common: Each of them is characterized by a progressive degeneration of nerve cells in certain parts of the body, whereas the six non-polyglutamine diseases are very heterogeneous in their manifestation. Below is a table with diseases, trinucleotide repeat and, if known, the number of repeats in affected individuals.

| | | Number of | |
|---|---|---|---|
| | Repeat | repeats | Number of repeats |
| Disease | sequence | if healthy | in affected |
| Dentatorubral | CAG | | 54-70 |
| pallidoluysian atrophy | | | |
| (DRPLA) | | | |
| Fragile X syndrome | CGG | 6-53 | 230 to >2,000 |
| Fragile site FRAXE | GCC | 6-35 | >200 |
| Huntington disease | CAG | | 35-121 |
| Kennedy disease | CAG | | >40 |
| Machado-Joseph | CAG | | 68-79 |
| disease | | | |
| Myotonic dystrophy | CTG | 5-37 | 50 to >2,000 |
| Friedrich's ataxia | GAA | 7-34 | >100 |
| Spinocerebellar ataxia | CAG | | 41-81 |
| type 1 | | | |
| Spinocerebellar ataxia | CAG | | |
| type 2 | | | |
| Spinocerebellar ataxia | CAG | | |
| type 3 | | | |
| Spinocerebellar ataxia | CAG | | |
| type 6 | | | |
| Spinocerebellar ataxia | CTG | 16-37 | >110 |
| type 8 | | | |
| Spinocerebellar ataxia | CAG | 7-28 | 66-78 |
| type 12 | | | |

All these repeat expansions are presently analysed with PCR followed by an electrophoresis step. This analysis is time-consuming and, for longer repeated sequences, there may be problems with sensitivity.

In a second embodiment the present invention relates to a method for analysing the number of nucleotide repeats in a nucleic acid sample, which method comprises the steps of:
a) providing a nucleic acid sample potentially comprising a nucleotide repeat
b) providing oligonucleotide(s) complementary to said nucleotide repeat
c) annealing said oligonucleotide(s) to said nucleic acid sample
d) ligating said oligonucleotide(s) annealed to said nucleic acid sample to each other using a ligase enzyme
e) detecting said ligation by-product to determine whether a ligation reaction has occurred,

wherein steps a)-e) are performed simultaneously, i.e. by mixing all components together, or subsequently, i.e. mixing only the components necessary for each step and adding additional components subsequently.

Two types of oligonucleotides are preferred for the analysing nucleotide repeats in accordance with the present invention. One preferred type of oligonucleotide is a single-, di- or multimer of the repeat unit in itself (i.e. the oligonucleotide consists of one or more repeats of the repeated sequence). The other preferred type is an “out of phase” oligonucleotide, that does not have a length equal to single-, di- or multi-mers (n-mers) of the nucleotide repeat unit, i.e. the oligonucleotide is shorter or longer than the n-mers of the nucleotide repeat unit in itself by one or more bases. The oligonucleotides in the “out of phase” case are added one at a time and allowed to ligate and the ligation by-product detected before the next oligonucleotide is added. It is preferable to initiate the start of the nucleotide repeat by the use of an oligonucleotide adapted to anneal immediately outside the repeated sequence, i.e. in one of the sequences flanking the nucleotide repeat. These different types of oligonucleotides and the different types of analysis they allow to be performed are described in more detail below.

Below some examples of oligonucleotides, suitable for the present invention, that can be used for calculation of the number of repeats in polyglutamine disorders are given:

| | |
|---|---|
| | Oligonucleotides |
| | which can be used in |
| CAGCAGCAGCAGCAGCAGCAGCAGCAG | detection of CAG repeats |

| | | |
|---|---|---|
| Variant 1 | GTCGTC etc. | pGT, pCG, pTC |
| Variant 2 | GTCGTCGTCGTC etc. | PGTCG, pTCGT, pCGTC |
| Variant 3 | GTCGTCGTCGTCGTC etc. | PGTCGT, pCGTCG, pTCGTC |
| Variant 4 | GTCGTCGTCGTCGTCGTCGTC etc. | PGTCGTCG, pTCGTCGT, |
| | | pCGTCGTC |

Variant 1 describes three different types of di-mers (normal font, bold and underlined, respectively) of oligonucleotide to analyse two repeated units.

Variant 2 describes three different types of 4-mers (normal font, bold and underlined, respectively) of oligonucleotide to analyse 4 repeated units.

Variant 3 describes three different types of 5-mers (normal font, bold and underlined, respectively) of oligonucleotide to analyse 5 repeated units.

Variant 4 describes three different types of 7-mers (normal font, bold and underlined, respectively) of oligonucleotide to analyse 7 repeated units.

Below some examples of oligonucleotides, suitable for the present invention, which can be used for calculation of the number of repeats in Fragile X Syndrome, are given:

| | |
|---|---|
| | Oligonucleotides |
| | which can be used in |
| CGGCGGCGGCGGCGGCGGCGGCGGCGG | detection of CGG repeats |

| | | |
|---|---|---|
| Variant 1 | GCCGCC etc. | pGC, pCG, pCC |
| Variant 2 | GCCGCCGCCGCC etc. | pGCCG, pCCGC, pCGCC |
| Variant 3 | GCCGCCGCCGCCGCC etc. | pGCCGC, pCGCCG, pCCGCC |
| Variant 4 | GCCGCCGCCGCCGCCGCCGCC etc. | pGCCGCCG, pCCGCCGC, |
| | | pCGCCGCC |

The enzymatic ligation reaction is performed by a number of different enzymes with ligase activity, of which preferred examples are described in detail below. By an enzyme having ligase activity is meant an enzyme that has the ability to form a phosphodiester bond between ends within a nucleic acid chain or between different nucleic acid chains. Recombinant variants of ligase enzymes are also contemplated in the present invention.

DNA-ligases fall into two broad classes (see Doherty and Suh, 2000, Nucleic Acid Res. 28, 21: 4051): those requiring ATP as cofactor and those requiring NAD⁺. The eukaryotic, viral and archaebacteria encoded enzymes all require ATP. However, since ATP is also a substrate in eukaryotic luciferase reactions (as described below), luciferase-based analysis has earlier been practically impossible to use to detect a ligation reaction when an ATP-dependent DNA-ligase is used. One possibility to use an ATP-dependent ligase would be (1) if by some means the excess of ATP can be reduced (such as by using apyrase), or (2) if an ATP-analogue (such as DATP) is used, which does not function as a substrate for luciferase, or functions as a substrate for luciferase to a significantly lower degree than ATP, or (3) a combination of (1) and (2).

In one embodiment, the present invention uses a NAD⁺-dependent DNA-ligase for ligating DNA-oligonucleotides annealed to a sample nucleic acid. Hereby, a DNA-ligase that is dependent on NAD⁺, instead of ATP, as an energy source, is used. NAD⁺-dependent DNA ligases are highly homologous, monomeric proteins of 70-80 kD found exclusively in eubacteria which catalyse the following reaction (wherein E is the ligase and pA is AMP, and p denotes a phosphate group):
E+NAD⁺<−>EpA+NMN⁺
EpA+pDNA<−>AppDNA+E
DNA_{OH}+AppDNA<−>DNApDNA+pA

T4-RNA-ligase is also preferably employed in the present invention when DNA, or RNA, or a combination of DNA and RNA-oligonucleotides are ligated to each other. For example, T4-RNA-ligases catalyse the following reaction for RNA-RNA ligation:
5′P−RNA+ATP→AdoPP5′RNA
AdoPP5′RNA+RNA3′OH→RNA−P−RNA+AMP

The first step in this reaction thus involves adenylation of RNA using ATP. However, since this, in some embodiments, is not preferred when one of the preferred analytical methods for detecting a ligation reaction is used, i.e. luciferase-based assays, an alternative is necessary. A number of researchers have reported synthesis of the AdoPP5′RNA-intermediate (e.g. Sninsky J J et al., The use of terminal blocking groups for the specific joining of oligonucleotides in RNA ligase reactions containing equimolar concentrations of acceptor and donor molecules (1976), Nucleic Acids Res. 3 (11) 3157-3166; England, T E et al., Dinucleoside pyrophosphates are substrates for T4-induced RNA ligase (1977), Proc. Natl. Acad. Sci USA 74 (11) 4839-4842). Thus a method has been devised to utilise such an intermediate (AdoPP5′RNA, or AdoPP5′DNA) directly in the method, thus avoiding the presence of ATP that would otherwise disturb the detection and T4-RNA-ligases are therefore also preferred for the present invention.

Another aspect, as an alternative method of ligation, is the use an ATP-dependent ligase in combination with an ATP-analogue, such as dATP, or 2-aminopurine riboside triphosphate. To function satisfactorily, the analogue must firstly support the ligation reaction. Secondly, it should ideally not function as a substrate in the luciferase reaction, at least not to the same degree as achieved by ATP, in order to minimise the background due to the presence of excess luciferase substrate. In addition, the product of the ligation reaction should preferably be pyrophosphate, which can then be converted by sulphurylase to ATP, which in turn can be detected by the luciferase reaction. Such a cofactor analogue would facilitate ligation detection above the low background signal resulting from the presence of excess analogue. An improved method might involve subsequent removal of the analogue itself prior to detection.

For example, experiments have demonstrated (see Kinoshita et al, J. Biochem 122 205-211 (1997)) that dATP is an effective cofactor for the ATP-dependent ligase T4 DNA ligase. Even dATP, however, gives a significant background signal with luciferase although this is lower than that caused by ATP. An alternative is to combine the use of dATP with post-ligation treatment with apyrase such that the excess dATP is degraded prior to detection of the light signal using luciferase. This combination has resulted in a surprisingly effective method for detecting, for example, variation in the length of DNA repeats, as described in the example section. The results are also comparable to those obtained using a NAD⁺-dependent ligase in combination with PPDK.

Further, since apyrase competes with the ATP-dependent ligase for the substrate (e.g. dATP), the concentrations of apyrase, ligase and substrate must be optimised for the specific situation. Thus, it is also possible to add apyrase before or during the ligation reaction, as well as after the ligation reaction, in order to reduce excess amounts of DNA ligase substrate.

Thus, in another embodiment the ligation step (step d) is performed employing a ATP-dependent ligase, and apyrase is added to the ligation mixture of step d) before, during or after ligation in order to reduce excess amounts of DNA ligase substrate.

In a preferred embodiment, the ATP dependent ligase is T4 DNA ligase.

In another preferred embodiment, dATP is used as a substrate for the ATP dependent ligase in the ligation step (step d).

As the last step of the method of the claims of the present invention a ligation by-product is detected to determine whether a ligation reaction has occured. The amount of ligation by-product produced is proportional to the number of ligations reactions that have occured. As described below, sometimes it is sufficient to detect whether or not a ligation reaction has occured in order to be able to estimate the number of nucleotide repeat units. A person skilled in the art may determine that further manipulation of the end product is desirable, and the present invention contemplates such an adjustment to the use of the method of the present invention.

Yet another embodiment of the present invention relates to a method for analysing the number of nucleotide repeats in a nucleic acid sample, which method comprises the steps of:
a) providing a nucleic acid sample potentially comprising a nucleotide repeat;
b) providing oligonucleotide(s) complementary to said nucleotide repeat;
c) annealing said oligonucleotide(s) to said nucleic acid sample;
d) ligating said oligonucleotide(s) annealed to said nucleic acid sample to each other using a ligase enzyme;
e) converting a ligation by-product into ATP; and
f) detecting said ATP to determine whether a ligation reaction has occurred,

wherein steps a)-f) are performed simultaneously, i.e. by mixing all components together, or subsequently, i.e. mixing only the components necessary for each step and adding additional components subsequently. Alternatively, some steps may be performed simultaneously and some steps subsequently. This can be preferable for example when the different steps require different conditions in order to be performed, e.g. different buffers, temperatures etc. All components necessary for the reaction except one can also be mixed prior analysis and the chain of reactions initiated by addition of the last component.

In this embodiment one of the ligation by-products (i.e. AMP or modified AMP or PPi (pyrophosphate) is converted into ATP.

In one preferred embodiment the ligation by-product is AMP.

AMP produced in a ligation reaction according to the present invention is preferably converted into ATP. In one preferred embodiment the AMP produced in a ligation reaction according to the present invention is converted into ATP by a one-step enzymatic reaction using pyruvate phosphate dikinase (PPDK) as follows.
AMP+PPi+phosphoenolpyruvate<−>ATP+Pi+pyruvate

The Kₘ for AMP is 250 × lower than the Kₘ for ATP. Thus the reaction is driven strongly in the direction of AMP→ATP. Pyruvate phosphate dikinase has a pH optimum of 6.5-7, a pH stability within the range of 6-11, an optimum temperature of 55-60° C., and is stable at temperatures below 55° C. Notable advantages of this enzyme is its stability and enhanced activity at higher temperatures (50-60° C.), which is an advantage since often temperatures in this range are preferred to provide annealing conditions with suitable stringency. Also, PPDK is used in buffers amenable to bioluminescent detection using firefly luciferase, a preferred detection assay described in detail below.

Alternatively, another by-product of a ligation reaction, namely PPi, is preferably converted to ATP. As discussed above, ATP-dependent DNA-ligases are in some embodiments not suitable for the present invention as the substrate in the ligation reaction, ATP, is the same as the substrate for the luciferase reaction (if not apyrase, for example, is used, as outlined above). However, DNA-ligases that can use a modified ATP-molecule, such as dATP or 2-aminopurine riboside triphosphate (Kinoshita, Y and Nishigaki, K. Unexpectedly general replaceability of ATP in ATP-requiring enzymes. J. Biochem 122 205-211 (1997)) are employed in the present invention. Preferred such DNA-ligases catalyse the following reaction (wherein A* denotes a modified adenosine-molecule as defined above):
E+pppA*<−>EpA*+PPi
EpA*+pDNA<−>A*ppDNA+E
DNA_{OH}+A*ppDNA<−>DNApDNA+A*p

One advantage of using a modified ATP-molecule is that the modified ATP-molecule does not interfere with a preferred luciferase-based assay (described below) to the same extent as ATP does and can therefore be present during the luciferase-based assay described below. The type and concentration of cofactor required by the ligase is a key factor for success in luciferase-mediated detection of ligation events. This cofactor must support effective ligation but not interfere with the detection step (if not apyrase, for example, is used, as outlined above).

The PPi produced in a ligation reaction according to the present invention is then preferably converted into ATP by the following reaction employing an ATP-sulfurylase (wherein APS denotes adenosine 5′-phosphosulfate):
PPi+APS→ATP+SO₄²⁻

In still another embodiment, the present invention relates to a method for analysing the number of nucleotide repeats in a nucleic acid sample, which method comprises the steps of:
a) providing a nucleic acid sample potentially comprising a nucleotide repeat;
b) providing oligonucleotide(s) complementary to said nucleotide repeat;
c) annealing said oligonucleotide(s) to said nucleic acid sample;
d) ligating said oligonucleotide(s) annealed to said nucleic acid sample to each other using a ligase enzyme;
e) converting a ligation by-product into ATP; and
f) detecting said ATP by a luciferase-based assay as a measure of whether a ligation reaction has occurred,

wherein steps a)-f) are performed simultaneously, i.e. by mixing all components together, or subsequently, i.e. mixing only the components necessary for each step and adding additional components subsequently. Alternatively, some steps may be performed simultaneously and some steps subsequently. This can be preferable for example when the different steps require different conditions in order to be performed, e.g. different buffers, temperatures etc. All components necessary for the reaction except one can also be mixed prior analysis and the chain of reactions initiated by addition of the last component.

The ATP produced, e.g. by any of the reactions described above for converting a ligation by-product into ATP, is preferably used as a substrate in a luciferase-based assay according to this embodiment. Since the amount of ATP is proportional to the amount of ligation by-product (AMP or PPi) produced, i.e. the number of ligation reactions that have taken place, and provided the amount of ATP is limiting for the luciferase reaction, i.e. luciferin and O₂ are present in excess, the light output is relative to the number of ligation reactions. The luciferases suitable for the present invention catalyze the following reaction:
ATP+luciferin+O₂→oxyluciferin+AMP+PPi+light

The wavelength of the emitted light depends on the origin of the luciferase employed.

In still another embodiment, the invention relates to a method for microbial typing of a nucleic acid sample, which method comprises the steps of:
a) providing a nucleic acid sample comprising at least one marker for microbial typing;
b) providing oligonucleotide(s) that are completely or partially complementary to the region(s) comprising marker(s) for microbial typing of said nucleic acid sample;
c) annealing said oligonucleotide(s) to said nucleic acid sample;
d) ligating said oligonucleotide(s) annealed to said nucleic acid sample to each other using a ligase enzyme; and
e) detecting a ligation by-product to determine whether a ligation reaction has occurred;
f) comparing the ligation pattern of the sample with a reference pattern, in order to determine the microbial type,

wherein steps a)-e) are performed simultaneously or subsequently or in any combination of subsequent steps.

Hereby, different species of DNA, such as from different microbial species, are resolved by choosing probes of suitable length and composition. For example, the most widely used target gene for comparative sequence analysis in bacteria is the 16S rRNA gene, which codes for the structural part of the 30S ribosomal small subunit. DNA sequencing of the 16S rRNA gene is an important tool for phylogenetic studies and has also been used for microbial identification.

All luciferase enzymes from all different organisms capable of catalysing the above reaction are contemplated in the present invention, including recombinant variants thereof. Thermoactive luciferases, including wild type enzymes from different species, mutant and recombinant variants, are available and can be used when it is preferable to perform the luciferase reaction at a higher temperature. The luciferase reaction can be performed following the ligation- and conversion-reactions by adding the reagents necessary for the reaction after these reactions have been allowed to occur. Alternatively, the reagents necessary for the luminescence reaction may be present simultaneously as the ligation and conversion-reactions are allowed to proceed to allow real-time measurement of ligation. In another embodiment a thermostable luciferase, which is active at a lower temperature such as luciferases described in Kajiyama N and Nakano E, 1993 (Thermostabilization of firefly luciferase by a single amino acid substitution at position 217 Biochemistry 32 (50 13795-9)) may be employed and the coupled enzymatic reactions performed in two steps: firstly the ligation and conversion-reactions are performed at a higher temperature, suitable for the specific application, and the temperature thereafter lowered and luciferase activity measured.

Native firefly luciferase is completely dependent on the presence of Mg²⁺, ATP, luciferin and molecular oxygen in the reaction mixture (see Ford, S R and Leach, F R, Improvements in the application of firefly luciferase assays. Methods in Molecular Biology, vol 102: Bioluminescence Methods and Protocols, pp 3-20, Ed R. A. LaRossa, Humana Press Inc. 1998). Dithiothreitol (DTT) and ethylenediaminetetraacetic acid (EDTA) are added to the reaction mixture to prevent inhibition of the reaction by oxidation of cysteine residues, or metal ions, respectively. The luciferase reaction is therefore preferably performed in a buffer comprising approximately 10 mM Mg²⁺ (e.g. Mg-acetate 10 mM) and DTT (e.g. 0.2 mM). The optimum temperature is 25° C. Temperatures above 30° C., and especially above 35° C. cause rapid inactivation of the wild type enzyme. The luciferase reaction is therefore preferably performed at pH 7.5-8, (more preferably pH 7.8), in the presence of 2-10 mM Mg²⁺, 0.5-2 mM DTT and 0.5-2 mM EDTA, at a temperature of 20-30° C. However, variants of luciferases may have other requirements regarding e.g. temperature as described above. Importantly, chloride ions may inhibit the luciferase reaction and it may therefore be suboptimal to have them present during the luciferase reaction.

There are many methods available for the detection of light as a result of a luminescence reaction, such as detection on photographic films, x-ray films, by the use of a photomultiplier tube (PMT) or charge coupled device (CCD) camera. For the present invention the use of a luminometric detection assay by PMT or CCD camera is preferable, since such methods allow for a quantitative measurement of light emission. A major advantage with the method provided in the present invention is that it allows a ligation reaction to be detected in real time.

Real-time detection of ligation could be improved further by the inclusion of an enzyme that ‘turns off’ the light production by the luciferin/luciferase reaction after the initial level has been detected. The “initial level” refers to the level of light that is necessary for the light release to be detected. This level is easily determined by the skilled person in relation to the detection apparatus that is used. This “turn off”-principle may also be of advantage with a low ‘burn rate’ of the luciferase enzyme. Such an enzyme would increase the turnaround time between ligation events. A suitable enzyme is apyrase, as described above, that would break down ATP to ADP, but not degrade AMP. This enzyme has been used to great advantage in conventional pyrosequencing (U.S. Pat. No. 6,258,568).

Thus, in yet another embodiment the light production in the luciferin/luciferase reaction is enzymatically turned off after an initial level of produced light has been reached. Preferably, this is made by the addition of apyrase.

The detection of a ligation by-product according to the method of the present invention gives two kinds of information: 1) if a ligation by-product is produced a ligation reaction has taken place. In some cases (as described below) this information is sufficient. 2) As mentioned above, the amount of ligation-by-product is proportional to the number of ligation reactions and can therefore be used to calculate the number of repeats of a repeated nucleic acid sequence, or markers for microbial typing, in a nucleic acid sample, provided the amount of nucleic acid sample is known.

The use of PPDK to convert AMP to ATP and then use the ATP to generate a luciferin-luciferase-based signal has been reported by Sakakibara et al. (Sakakibara T et al., Anal Biochem. 1999 268(1):94-101) and Ito et al (Ito et al., Anal. Sci. 2003, 19:105-109)). However, in these applications the method was used either to measure the AMP produced by degradation of RNA to provide an index for hygiene monitoring of foodstuffs (Sakakibara et al, 1999), or in a dual immunological reaction (Ito et al, 2003), respectively.

A number of ribozymes have also been reported to have ligase activity and are also contemplated in the present invention (Glasner, M E et al (2002) Metal ion requirements for structure and catalysis of an RNA ligase ribozyme, Biochemistry, 41, 8103-8112; McGinness, K. E. and Joyce, G. F. (2002) RNA-catalysed RNA ligation on an external RNA template, Chemistry and Biology 9, 297-307). Ligation may involve the ribozyme directly ligating to a substrate (e.g. class I ligase, Glasner et al 2002), and even ligation of two oligonucleotides that are bound at adjacent positions on a complementary template (hc ligase ribozyme, McGiness and Joyce, 2002). In each case, ligation involves joining the 3′ —OH group of one molecule to the 5′-triphosphate of another molecule and the release of pyrophosphate. No cofactor such as ATP is involved in the reaction and ribozymes are therefore suitable for the present invention when the preferred detection of a ligation reaction involves the use of luciferase-based assays.

In addition to the elsewhere in the present disclosure mentioned ligase enzymes, suitable ligase enzymes for the present invention are exemplified, but not limited to, the following ligase enzymes: ATP-dependent ligase enzymes such as T4 DNA ligase, T7 DNA ligase, Bst DNA ligase, Tfi DNA Ligase, NAD⁺-dependent ligase enzymes, such as Tth DNA ligase, Tfl DNA ligase, Tsp DNA ligase, E. coli DNA ligase, and RNA ligase enzymes, such as tRNA ligases.

Two types of analysis are possible when the method of the present invention is performed. The number of repeats in a specific nucleic acid sample can be determined by constructing a standard curve of detection of a ligation by-product by using a known amount of template DNA which has a known number of repeated sequences. Since each ligation reaction results in one ligation by-product, determination of the amount of ligation by-product allows the number of nucleotide repeats to be determined. Provided the amount of template DNA in the nucleic acid sample to be analyzed is known, the number of repeats can be determined by relating the amount of ligation by-product to the number of repeats using the standard curve.

In another preferred embodiment “out of phase”-oligonucleotides are used as described above. In this case a ligation by-product is produced which is detectable, e.g. as described above, at each addition of an additional oligonucleotide until the whole stretch of template DNA is filled with ligated oligonucleotides. Therefore, in this case, the amount of template does not have to be known. If the repeat unit is 2, each out of phase oligonucleotide must have 3 or more nucleotides with an uneven number. If the repeat number is 3 the oligonucleotide can be 2 nucleotides or more, up to the total length of the sequence of repeated units, except for multiples of 3. For example, for detection of triple repeats, combinations of 4, 5, 7, 8, 10, 11-mers, etc could be used. When out of phase oligonucleotides are used, an oligonucleotide which is adapted to anneal immediately outside the nucleotide repeat sequence is preferably added first. Importantly, the oligonucleotides have to be constructed so that each oligonucleotide only can ligate in one place (i.e. next to the oligonucleotide added in the ligation cycle immediately preceding the present ligation cycle).

When out of phase oligonucleotides are used, unannealed oligonucleotides (i.e. oligonucleotides which are not annealed or ligated) from one ligation cycle are preferably removed before the next ligation cycle is started. This is achieved in different ways. One way is to use a single stranded-DNA dependent exonuclease that digests unannealed oligonucleotides that are present during the ligation reaction. For example, exonuclease I (Exo I), the product of the sbcB gene of E. coli, is an exodeoxyribonuclease that digests single-stranded (ss) DNA in a 3′ to 5′ direction. Enzymatic activity absolutely requires the presence of a free 3′-hydroxyl terminus. In this case it is necessary to protect the template by modification of the 3′-end, such as by incorporation of a dideoxy nucleotide using terminal deoxynucleotidyl transferase. Alternatively, in another preferred embodiment of the present invention an enzyme is added during or after the ligation reaction step and which inactivates the unannealed oligonucleotides by removing the 5′ phosphate from the oligonucleotide. This enzyme can be a phosphatase or a 5′ end specific exonuclease dependent on a 5′ end phosphate. The phosphatase can be selected from the group of shrimp alkaline phosphatase SAP, calf intestine alkaline phosphatase CIP or any bacterial alkaline phosphatase. It is easily realized by the skilled person that removal of unannealed oligonucleotides as described above also can be utilized when n-mers of the repeat unit are used as oligonucleotides.

In another preferred embodiment of the present invention the nucleic acid sample is immobilized on a support. The immobilization can be either covalent or non-covalent. A common non-covalent way used in the art is the utilization of a biotin-streptavidin binding pair, where the biotin is bound to the primer and the streptavidin is bound to the support. A frequently used covalent binding method is the use of an amino-linker attached to the primer, which easily reacts with an epoxy-silane treated surface. The support can be of several types, beads (solid or porous), or the surface of a chip or fiber. An alternative is to bind the primer that is complementary to the flanking sequence of a region comprising genetic elements, such as a nucleotide repeat, or one marker for microbial typing, to a support, via for example a biotin-streptavidin binding pair. One advantage with the use of an immobilized nucleic acid sample or primer is that excess enzymes, oligonucleotides and/or degradation products, in one preferred embodiment, easily can be removed after one ligation-detection cycle before the next is initiated, by a washing step.

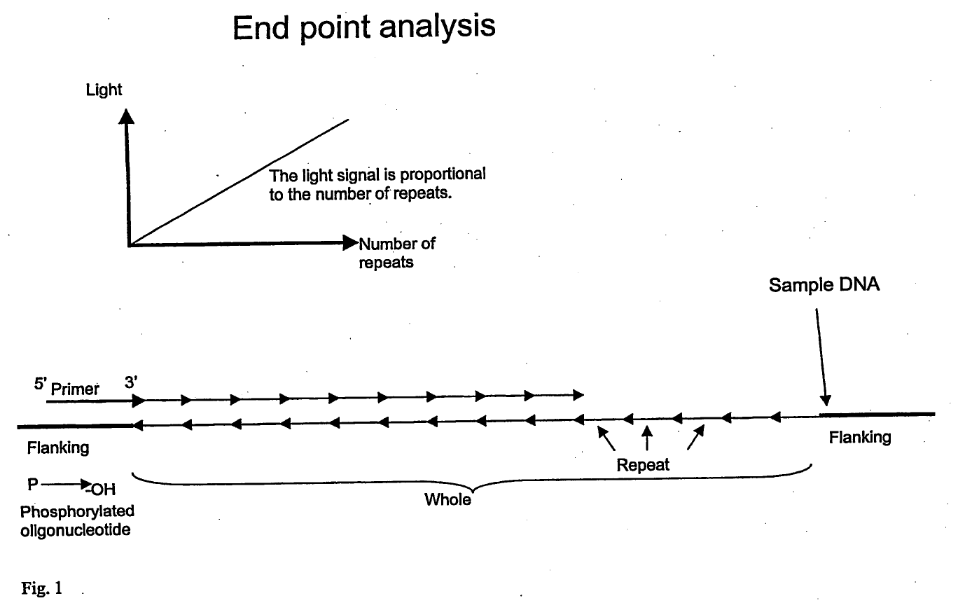

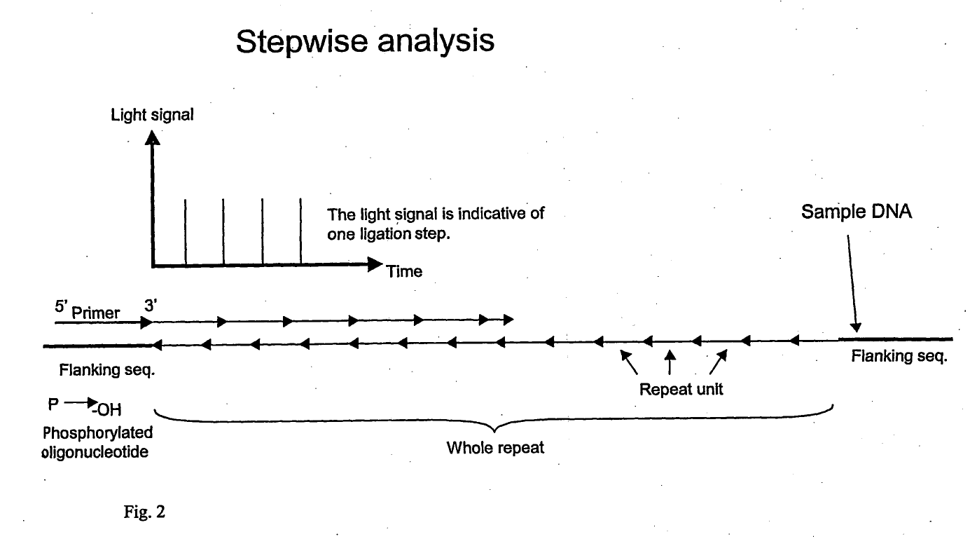

The two types of analysis described above are exemplified in FIGS. 1 and 2 with a variant of the present invention when the ligation by-product is converted into ATP which is used in a luciferase-based assay to detect the occurrence of a ligation reaction. FIG. 1 shows an “end-point”-analysis in which the oligonucleotide which is a single- or multimer of the repeat unit in itself is used as an oligonucleotide. Each ligation reaction results in a certain amount of light emitted, the total of which is quantified. The amount of light emitted can be related to the number of repeats provided the amount of sample nucleic acid is known using a standard curve as described above. In FIG. 2, out of phase oligonucleotides are used which are subsequently added, allowed to ligate and the light emitted in one ligation reaction detected in a “step-wise” fashion before the next oligonucleotide is added and the cycle is started all over again (i.e. addition, ligation and detection of light emission). This is repeated until no more light is emitted, i.e. as is the case when the whole stretch of the repeated sequence in the sample nucleic acid is filled with ligated oligonucleotides. Thereby the number of repeat units in the repeated sequence can be determined without knowing the amount of sample nucleic acid.

Depending on choice of oligonucleotide length and sequence the method can be used for repeat sequences in the range of two to several thousand bases.

The ligation reaction is performed over a wide range of temperatures, depending on the choice of ligase and the type of nucleic acids that are to be ligated. For example, when oligonucleotide probes are used which have a high degree of complementarity to the template nucleic acid to which they are to be hybridized, a higher temperature can be used during the ligation reaction, by employing a heat-stable ligase, than if the complementarity is low. The choice of buffer and the concentration of ligase during the ligation reaction have to be optimized for each specific ligase and is within the ability of a skilled person.

In a preferred embodiment, the ligation reaction is performed using a NAD⁺-dependent DNA-ligase and the AMP ligation by-product produced is transformed into ATP using a PPDK-enzyme. The ATP produced is then preferably detected using a luciferase-based assay.

In a preferred embodiment of the present invention, a thermostable ligase (e.g. Taq DNA ligase) is used to cycle the ligation reaction independent of the detection step (which involves thermal-labile reagents) and the product of the reaction is transferred to the detection reagents. Alternatively the reaction is cooled and the thermal-labile components are then introduced into the reaction chamber.

In one embodiment of the present invention a PCR-reaction is performed on the nucleic acid sample to amplify the amount of target nucleic acid before the ligation reaction. This is preferred if the amount of template nucleic acid to be analyzed for nucleotide repeats is low. Also this allows an allele specificity to be obtained if PCR-primers that are specific to an allele specific sequence adjacent to the repeated sequence are used.

In organisms such as humans it is very common to find different allelic forms of two alleles of a nucleotide repeat, which is frequently used in forensics to solve crimes. The present invention will solve a situation with different numbers of repeats in the two alleles, since the amount of ligation by-product is proportional to the number of ligation reactions. When the shorter allele is filled with oligonucleotide probes, the number of ligation-by-products will be reduced by 50%. For example, when the ligation reactions are detected via emitted light using a luciferase-based assay as described above, the light output will be reduced by approximately 50%, a difference that can easily be monitored by luminometry. Therefore the number of repeat units in each allele can be determined by the present invention.

The present invention is also related to a kit for performing the methods according to the present invention which kit comprises, in separate vials, a ligase enzyme and an enzyme for converting a ligation by-product into ATP. Preferably the ligase enzyme is a NAD⁺-dependent DNA-ligase. Preferably the enzyme for converting a ligation by-product is a PPDK-enzyme. The invention also relates to a kit further comprising, in a separate vial, a luciferase enzyme. A kit according to the present invention may preferably comprise oligonucleotides complementary to a region comprising a genetic element, such as a nucleotide repeat, optionally with a AdoPP5′ modification, associated with a disease selected from the following group of diseases: Dentatorubral pallidoluysian atrophy (DRPLA), Fragile X syndrome, Fragile site FRAXE, Huntington's disease, Kennedy's disease, Machado-Joseph disease, Myotonic dystrophy, Friedrich's ataxia, Spinocerebellar ataxia type 1, Spinocerebellar ataxia type 2, Spinocerebellar ataxia type 3, Spinocerebellar ataxia type 6, Spinocerebellar ataxia type 8 and Spinocerebellar ataxia type 12. Moreover, in one embodiment the kit further comprises, in a separate vial, apyrase.

Another kit according to the present invention may preferably comprise oligonucleotides complementary to a genetic region, optionally with an AdoPP5′ modification, that is informative for identification of microbial species, from the following group: the 16S rRNA gene, 23S rRNA gene, groEL, gyrB, rpoB, rnpB and groEL, microsatellite and minisatellite sequences, VNTRs, the nuclear ribosomal DNA (rDNA) array—small-subunit (SSU) (18S-like), large-subunit (LSU)(23S, 26S, or 28S-like), 5.8S rRNA genes, and internal transcribed ribosomal DNA (rDNA) spacers (ITS1 and ITS2).

The vials in the kits according to the present invention can also comprise two or more of the components of the kit, provided the kit comprises at least two vials.

The present invention is also related to a composition for performing the method according to the present invention which composition comprises a ligase enzyme and an enzyme for converting a ligation by-product into ATP. Preferably the ligase enzyme is a NAD⁺-dependent DNA-ligase. Preferably the enzyme for converting a ligation by-product is a PPDK-enzyme. The invention also relates to a composition further comprising a luciferase enzyme. A composition according to the present invention may preferably comprise oligonucleotides complementary to a region comprising a genetic element, such as a nucleotide repeat, optionally with a AdoPP5′ modification, associated with a disease selected from the following group of diseases: Dentatorubral pallidoluysian atrophy (DRPLA), Fragile X syndrome, Fragile site FRAXE, Huntington's disease, Kennedy's disease, Machado-Joseph disease, Myotonic dystrophy, Friedrich's ataxia, Spinocerebellar ataxia type 1, Spinocerebellar ataxia type 2, Spinocerebellar ataxia type 3, Spinocerebellar ataxia type 6, Spinocerebellar ataxia type 8 and Spinocerebellar ataxia type 12.

Another composition according to the present invention may preferably comprise oligonucleotides complementary to a genetic region, optionally with an AdoPP5′ modification, that is informative for identification of microbial species, from the following group: the 16S rRNA gene, 23S rRNA gene, groEL, gyrB, rpoB, rnpB and groEL, microsatellite and minisatellite sequences, VNTRs, the nuclear ribosomal DNA (rDNA) array—small-subunit (SSU) (18S-like), large-subunit (LSU)(23S, 26S, or 28S-like), 5.8S rRNA genes, and internal transcribed ribosomal DNA (rDNA) spacers (ITS1 and ITS2). U.S. Pat. No. 4,988,617 describes a method for analysing SNPs which involve the detection of a ligation reaction using specific oligonucleotides designed such that the 3′-end of the ‘upstream’ oligonucleotide is placed over the polymorphic position. The ‘downstream’ oligonucleotide is placed with its 5′-end juxtaposed to the upstream primer. The 5′-end of the downstream primer is phosphorylated to permit ligation to the 3′-OH end of the downstream oligonucleotide. One version of the upstream primer is complementary at its 3′ end with a particular template molecule, whilst it will form a mismatch with a template molecule of the alternative genotype. This mismatch will hinder the ligation reaction due to the specificity of the DNA ligase, and hence the ligation or lack of ligation can be used to detect the polymorphism. The probes are then separated from the target nucleotide sequence and the presence or absence of a ligated product detected. This detection can involve separation of nucleotide stretches on a gel by using immobilised probes.

In another preferred embodiment of the present invention, the present method is used to analyse SNPs. A majority of mutations in the human genome are single nucleotide polymorphisms, SNPs, and they can often be linked to genetic diseases. Therefore, it is of importance to have reliable methods to detect such SNPs. Currently available methods for detecting SNPs are methods which depend on different denaturation of mismatched probes, such as in allele specific oligonucleotide hybridization (Wallace, R B et al, 1979, Nucl. Acid Res. 6: 3553) and denaturing gradient gel electrophoresis (Myers, R M et al, 1985, Nature 313: 495). Alternatively restriction fragment length polymorphism (RFLP) (Geever et al, 1987, Proc. Natl. Acad. Sci. 78: 508) can be used in which alternative digestion patterns are detected which are the result of differences in nucleotide sequences. However, only approximately one third of the nucleotides in the human genome can be analysed by this technique. RFLP allows approximately half of the SNPs to be analysed. The present invention can be used to analyse SNPs in any DNA-containing sample, such as a blood, cell or tissue from any plant, animal or microbe. Oligonucleotides are constructed which anneal immediately adjacent to each other on a template DNA. The oligonucleotides are constructed so that the end nucleotide of one of the oligonucleotides, at the end, which is adjacent to the other probe, has a nucleotide that is complementary to either the normal or the abnormal nucleotide of the nucleotide of the template nucleic acid at that position. The oligonucleotides are allowed to anneal to the template nucleic acid, whereafter the steps of the method of the present invention are allowed to take place as described above. If the nucleotide at the polymorphic site of the template nucleic acid is complementary to the nucleotide in this position of the oligonucleotide, a ligation reaction will take place with the release of a ligation-by-product, which can be converted to ATP for producing a light signal, which finally can be detected. If there is no complementarity between the oligonucleotide and the template nucleic acid no ligation will occur and accordingly no light emission will take place. Thereby the identity of the nucleotide at the polymorphic site can be determined.

The present invention is also suitable for determining telomere length, which is a nucleotide repeat of about 8-10 nucleotides. It has been suggested the ageing of humans is related to shortening of the telomeres and different forms of cancers might also be dependent of shorting of telomeres.

The present invention is also suitable for determining the length of microsatellites, which are frequently used in different forensic and paternity tests. The most frequently used microsatellites are CA repeats.

The method of annealing contiguous probes and then detecting which probes have annealed by a ligation event linked to a luciferase reaction can be applied to many other types of genetic analysis. One example is the identification of bacteria, for which molecular methods are frequently based on the amplification of a target sequence fragment. The amplicon is then analyzed to get a yes/no answer (DNA-hybridization, species specific PCR) or a pattern (fragment polymorphism, DNA sequencing) that can be associated with a specific strain, species or group. The most widely used target gene for comparative sequence analysis in bacteria is the 16S rRNA gene, which codes for the structural part of the 30S ribosomal small subunit. DNA sequencing of the 16S rRNA gene is an important tool for phylogenetic studies and has also been used for microbial identification. The 16S gene is rather large (˜1500 bases) and often contains too little variation for classification of closely related strains. Furthermore, in many genera it is a multi-copy gene, which may lead to sequence heterogeneity. Because of these disadvantages, additional target genes have been investigated including groEL, gyrB, rpoB, rnpB and groEL. The 23S gene is also becoming a suitable alternative. Microsatellite and minisatellite sequences, involving repeats in the range 1-10 base pairs and 10-100 base pairs, respectively, are also used in bacterial identification. Many are referred to as VNTRs (Variable number of tandem repeats). Furthermore, among eukaryotic organisms, such as fungi, the nuclear ribosomal DNA (rDNA) array—small-subunit (SSU) (18S-like), large-subunit (LSU)(23S, 26S, or 28S-like), 5.8S rRNA genes, and internal transcribed ribosomal DNA (rDNA) spacers (ITS1 and ITS2)—provides an ideal target for molecular identification. In fungi, embedded expansion segments (ES) regions of the 23S-like gene are most variable and hence serve as the diagnostic target sequence. DNA and RNA viruses may be detected and typed using a number of specific genetic regions e.g. L1, E6, E6/E7, E7/E1, and E1 regions for typing human papillomavirus (HPV). The variable regions mentioned above may be suitable targets for analysis by a ligation-mediated method.

The present invention is therefore also suitable for determining the length of nucleotide repeats, or otherwise characterising genetic variability, in microbial species, which is a useful tool for identification of strains and isolates.

Further, in one embodiment of the invention, the signal obtained from the ligation reaction(s) is normalised/calibrated, in advance to performing the methods of the present invention, by using a signal generated by a separate ligation or primer-extension event performed at another position on the template molecule or on a representative alternative DNA strand.

Also, in another embodiment different DNA strands to be analysed are included in the same reaction and suitable ligatable probes are added in order to analyse the strands in parallel or sequentially in a multiplex reaction.

Below, the invention will be described by way of examples, which examples are intended to illustrate the invention and not to limit the scope of the invention in any way.

### EXAMPLES

### Example 1

### Ligation of Two Oligonucleotides at a Variable Position in a Gene with a Thermocycled Ligation Reaction Followed by Bioluminescent Detection

Oligonucleotides used

| | | |
|---|---|---|
| Name | 5′-3′ | Modification |

| | | |
|---|---|---|
| BGL-1 | ATGGTGCACCTGACTCCTGA | 5′ biotin |
| BGL-2 | GGAGAAGTCTGCCGTTACTGC | 5′ P |
| BG-T | GCAGTAACGGCAGACTTCTCCTCAGGAGTCAGGTGCACCAT | |
| Complete upper strand | ATGGTGCACCTGACTCCTGAGGAGAAGTCTGCCGTTACTGC | |
| BG-S1 | ACGGCAGACTTCTCC | |

The oligonucleotides were designed to represent a synthetic version of the region of the beta-globin gene containing a point mutation causing sickle-cell anaemia (see Barany, F. (1991) Genetic disease detection and DNA amplification using cloned thermostable ligase. Proc. Natl. Acad. Sci. USA 88, 189-193).

The oligonucleotides form the following complex after annealing:

| | |
|---|---|
| | BGL-2 |
| BGL-1 | |
| ---------------------------- | A==========================-Biotin |
| ---------------------------- | T-------------------------- |
| | BGL-T |

The following were mixed in a final volume of 50 μL in a 200 μL PCR tube: 20 mM Tris-acetate buffer, pH 7.6; 10 mM magnesium acetate; 10 mM dithiothreitol; 1 mM NAD⁺; 20 U Taq DNA ligase (New England Biolabs); 0, 2, 4 or 10 pmole of template BGL-T, 40 pmol of each of the oligonucleotides BGL-1 and BGL-2. Controls were included that omitted the ligase enzyme. The tube was incubated in a thermocycler (MJ Research Tetrad) using the following method:
(94° C., 1 min; 65° C., 4 min)×10; 8° C.

The success of the ligation was confirmed in parallel experiments involving capture of the ligated product (biotin-BGL1-BGL2) on Streptavidin-Sepharose beads followed by denaturation, annealing of the specific sequencing primer BG-S1, and confirmation of the complete sequence by Pyrosequencing.

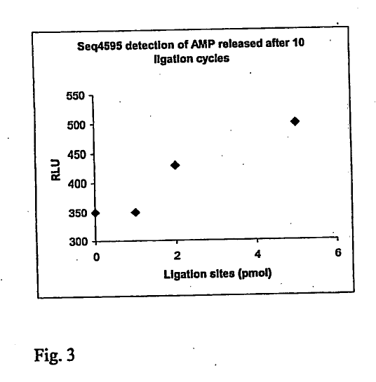

Twenty-five microlitres of the reaction were transferred to a PSQ96 plate. Twenty microlitres of Detection Mix#1 were added, containing 20 mM Tris-acetate buffer, pH 7.6; 10 mM magnesium acetate; 0.625 mM phosphoenolpyruvate; 0.375 mM sodium pyrophosphate; 50 μg PPDK. The plate was transferred to a PSQ96 Pyrosequencing Instrument where 5 μL of Detection Mix#2 (20 mM Tris-acetate buffer, pH 7.6; 10 mM magnesium acetate; 13 μg luciferase; 7.5 μg luciferin) were added using the dispensing cassette of the PSQ96 instrument, with an incubation temperature of 37° C. The development of light was followed using the CCD camera in the PSQ96 instrument. The results for 0, 2, 4 and 10 pmoles of the template BGL-T in the original reaction are shown in FIG. 3.

The signal clearly increases with the amount of template in the reaction mix, indicating that the linked ligation and conversion of released AMP to ATP and then to light has worked.

### Example 2

### Ligation of Two Oligonucleotides at a Variable Position in a Gene with the Ligation Reaction Linked to Bioluminescent Detection. Combination of Ligase and PPDK Steps on Pre-Annealed Primer/Template Complex

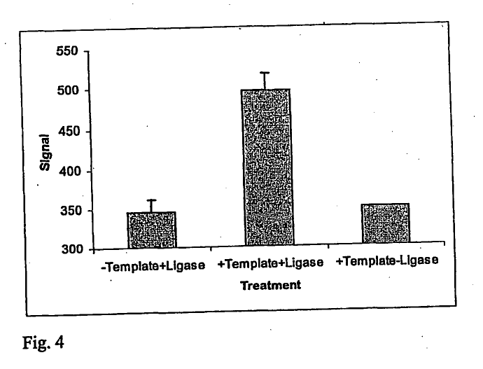

The following were mixed in triplicate wells in a final volume of 20 μL in a 200 μL PCR tube: 20 mM Tris-acetate buffer, pH 7.6; 10 mM magnesium acetate; 5 pmole of template BGL-T; 20 pmol of each of the oligonucleotides BGL-1 and BGL-2. The oligonucleotides were annealed to the template by incubating at 80° C. for 5 minutes followed by cooling to room temperature. The annealed reaction was transferred to a microtitre plate used in PSQ96. Controls received only buffer. Twenty microlitres of a mix containing reagents for ligation and conversion of released AMP to ATP were then added. This mix contained the following: 20 mM Tris-acetate buffer, pH 7.6; 10 mM magnesium acetate; 2.5 mM NAD⁺; 25 mM DTT; 0.625 mM phosphoenolpyruvate; 0.375 mM sodium pyrophosphate; 50 μg PPDK; 20 U Taq DNA ligase (omitted in controls). The reaction was incubated at 45° C. for 10 minutes. The plate was transferred to a PSQ96 and 5 μL of Detection Mix#2 (20 mM Tris-acetate buffer, pH 7.6; 10 mM magnesium acetate; 13 μg luciferase; 7.5 μg luciferin) were added using the dispensing cassette of the PSQ96 instrument, with an incubation temperature of 28° C. The development of light was followed using the CCD camera in the PSQ96 instrument (see FIG. 4).

### Example 3

### Detection of Difference in Number of CTG Repeats

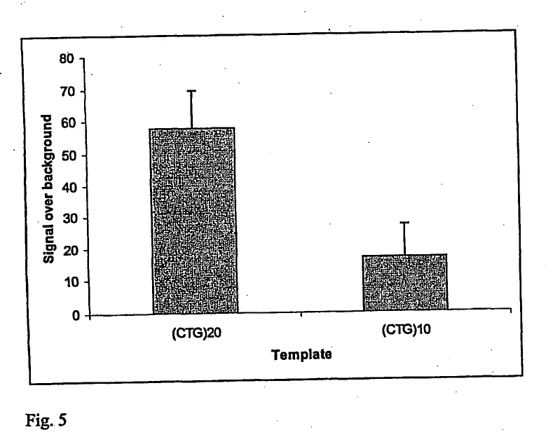

The experiment was based on the trinucleotide repeat (CAG/CTG) that is involved in a number of polyglutamine diseases (see table above). One picomole of oligonucleotide template with the sequence (CTG)₂₀ or (CTG)₁₀ was mixed with 40 picomoles of the complementary 5′-phosphorylated oligonucleotide, (CAG)₃ in 20 μL of Annealing Buffer (20 mM Tris-acetate, pH 7.6; 10 mM magnesium acetate; 20 mM potassium acetate) in a 96-well PSQ96 Plate. The short, phosphorylated oligonucleotide was annealed to the longer oligonucleotide templates by incubating for 5 minutes at 80° C. and then allowing to cool to room temperature. Ligation was performed by adding 5 μL of Ligation Mix (20 U Taq DNA ligase, 6.25 mM NAD⁺, and 62.5 mM dithithreitol in Annealing Buffer) and incubating for 30 minutes at 45° C. Controls with Ligation Mix without ligase were also run. The AMP released by the ligation reaction was converted to ATP by adding 15 μL of PPDK mix (50 μg PPDK, 0.8 mM PEP, and 0.5 mM sodium pyrophosphate in Annealing Buffer) and incubating for 10 minutes at 45° C. To determine the amount of ATP produced, the PSQ96 Plate was transferred to a PSQ96 Pyrosequencing Instrument where 5 μL of Detection mix (13 μg luciferase and 7.5 μg luciferin in Annealing Buffer) were dispensed by the instrument and the resulting light release was detected. The results for quadruplicates, with signals from controls (without ligase) subtracted, are shown in FIG. 5. These results clearly show an increase in signal when the number of CTG repeats increases (FIG. 5).

### Example 4

### Use of Out of Phase Oligonucleotide for Determination of the Number of Nucleotide Repeats

This example describes how a repeated sequence in a sample DNA is analysed with stepwise ligation steps of oligonucleotides longer or shorter than (i.e. out of phase) the repeated unit to be analysed by the present invention.

Firstly a primer-template complex is formed by adding a oligonucleotide that is complementary to the sequence flanking the nucleotide repeat sequence under conditions that allow annealing to occur:

Primer
Flanking sequence-
CGGCGGCGGCGGCGGCGGCGGCGGCGGCGGCGGCGG-
flanking sequence

Secondly a phosphorylated oligonucleotide longer (or shorter) than the repeated unit (three in this case) is added together with a ligase and ligation cofactor in a suitable ligation buffer.

Add: pGCCG + ligase
Primer
Flanking sequence-
CGGCGGCGGCGGCGGCGGCGGCGGCGGCGGCGGCGG-
flanking sequence

The ligation by-product released in the ligation reaction is then detected as an indication of that a ligation reaction has occured. For example by conversion of released AMP or PPi into ATP which can be detected in a luciferase-based assay as described above.

The excess of phosphorylated oligonucleotide is then remove before the next phosphorylated oligonucleotide (pCCGC), together with ligase and ligation cofactor, as described above, is added and another round of annealing, ligation and detection is initiated.

Add: pCCGC + ligase
Primer GCCG
Flanking sequence-
CGGCGGCGGCGGCGGCGGCGGCGGCGGCGGCGGCGG-
flanking sequence

This cycle of annealing ligation and detection is then repeated until the whole stretch of nucleotide repeat is filled with ligated oligonucleotides. Since the number of ligation reactions that have taken place are known, the number of repeat units in the template nucleic acid can be calculated.

### Example 5

### Use of ATP-Dependent Ligase in Combination with Apyrase-Mediated Removal of Excess Cofactor

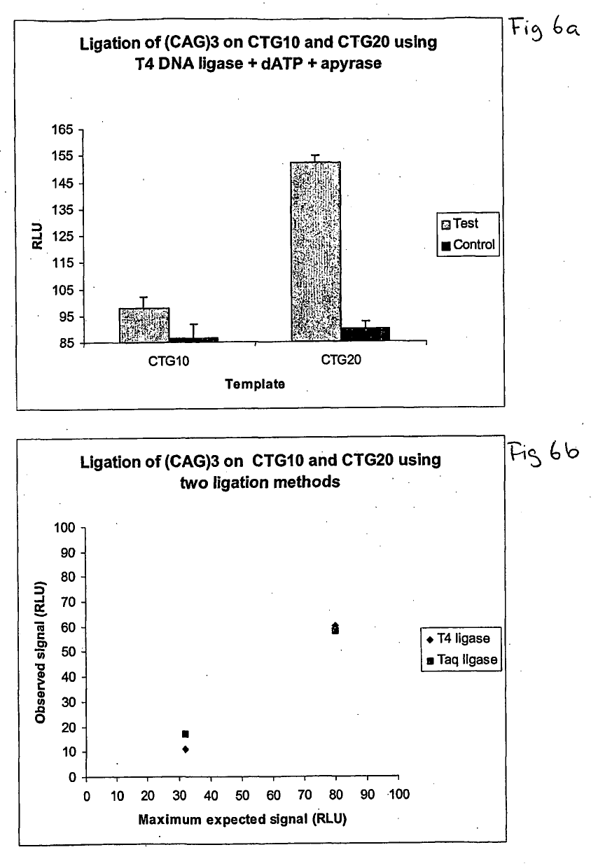

The experiment was based on the trinucleotide repeat (CAG/CTG) that is involved in a number of polyglutamine diseases (see table above). One picomole of oligonucleotide template with the sequence (CTG)₁₀ or (CTG)₂₀ was mixed with 40 picomoles of the complementary 5′-phosphorylated oligonucleotide, (CAG)₃ in 15 μl of Annealing Buffer (20 mM Tris-acetate, pH 7.6; 2 mM magnesium acetate) in a 96-well PSQ96 Plate. The short, phosphorylated oligonucleotide was annealed to the longer oligonucleotide templates by incubating for 5 minutes at 80° C. and then allowed to cool to room temperature. Ligation was performed by adding 15 μl of Ligation Mix (200 U T4 DNA Ligase, 2 mM dATP, and 2 mM dithithreitol in Annealing Buffer) and incubating for 30 minutes at 37° C. Controls with Ligation Mix without ligase and (CAG)₃ were also run. Twenty-five microliters of the ligation reaction were treated with apyrase to digest excess dATP by adding 50 mU apyrase in 15 μl Annealing Buffer and incubating at room temperature (c. 25° C.) for 25 minutes. To determine the amount of pyrophosphate produced by the ligation reaction, the PSQ96 Plate was transferred to a PSQ96 Pyrosequencing Instrument where 5 μl of Enzyme mix (25 mU sulphurylase and 0.5 μg luciferase in Annealing Buffer) and 5 μl of Substrate Mix (280 pmol APS and 7.5 μg luciferin in Annealing Buffer) were displaced by the instrument and the resulting light emission was detected. The signal obtained from known amounts of pyrophosphate and ATP was then determined by dispensing 5 picomoles of pyrophosphate into each well, followed by 5 picomoles of ATP. The results for triplicates, with signals from controls (without ligase and phosphorylated oligonucleotide), are shown in FIG. 6a.

The results in FIG. 6a shows clearly that the signal obtained from T4 DNA ligase in combination with dATP is dependent on the length of the DNA repeat region, with good resolution of the ligation signal (Test) over background, as determined in the absence of ligated oligonucleotide (Control).

In FIG. 6b, the results obtained (“Observed signal”) from NAD⁺-dependent Taq DNA ligase+PPDK (Example 3) and ATP-dependent T4 DNA ligase (using dATP as substrate, current example) are compared with the signal that could be obtained from the maximum number of ligations per template molecule (calculated from a theoretical maximum number of ligations and the signals obtained from 5 pmole of pyrophosphate—“Maximum expected signal”). The results indicate clear correlations (1) between expected and observed results, and even (2) between two independent ligation methods.

### Example 6

### Microbial Typing

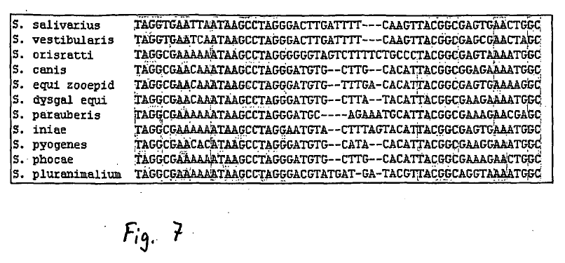

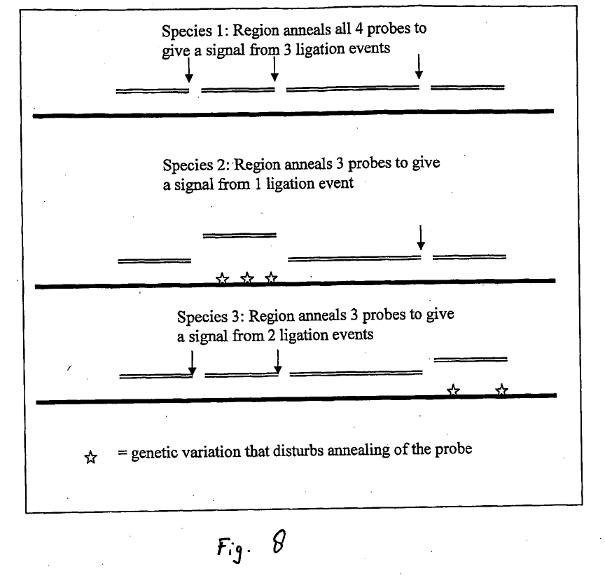

This example demonstrates how the ligation method may be used to type bacteria. FIG. 7 shows an alignment of sequences from a region of the rnpB gene from a number of species of streptococci. As can be seen, there are both common regions (shaded) and regions that vary between species. Ligation-mediated detection of different species or species groups is performed as follows. Probes of different lengths and compositions is designed such that they can be annealed, either simultaneously or successively, to contiguous parts of such a region with high or low efficiency depending on the species (see FIG. 8). The level of annealing is controlled by applying stringent conditions e.g. with temperature, salt concentration, or other agents such as dimethylsulfoxide that are known to a person skilled in the art The success of annealing is monitored by a ligation reaction such that the signal obtained is an indication of how many probes have annealed, and thus gives an indication of identity of the template. The annealing and detection steps are carried out either using all probes simultaneously, or by applying probes successively, with subsequent detection, depending on the application. This can be used as a simple method for screening to identify different groups of species etc., before analyzing in detail by sequencing if necessary. The principle can of course be applied to any genetic variation involving several bases and is not limited to microbial typing. The method may further include probes in a conserved region that can always be expected to anneal and thus give a signal that (1) confirms that the reactions have worked, and (2) provides a standard signal to normalize the subsequent signals from other ligation events.

1. Method for determining the presence of a genetic element such as a nucleotide repeat or a marker for microbial typing in a nucleic acid sample, which method comprises the steps of:
a) providing a nucleic acid sample comprising a genetic element;
b) providing an oligonucleotide that is completely or partially complementary to a region comprising the genetic element of said nucleic acid sample;
c) annealing said oligonucleotide to said nucleic acid sample;
d) ligating at least two of said oligonucleotides annealed to said nucleic acid sample to each other using a ligase enzyme; and
e) detecting a ligation-by-product to determine whether a ligation reaction has occurred, as a measure of the presence of the genetic element,
wherein steps a)-e) are performed simultaneously or subsequently or in any combination of subsequent steps.

2. Method for analysing the number of nucleotide repeats in a nucleic acid sample, which method comprises the steps of:
a) providing a nucleic acid sample potentially comprising a nucleotide repeat;
b) providing an oligonucleotide complementary to said nucleotide repeat;
c) annealing said oligonucleotide to said nucleic acid sample;
d) ligating at least two of said oligonucleotides annealed to said nucleic acid sample to each other using a ligase enzyme; and
e) detecting a ligation by-product to determine whether a ligation reaction has occured,
wherein steps a)-e) are performed simultaneously or subsequently or in any combination of subsequent steps.

3. Method for analysing the number of nucleotide repeats in a nucleic acid sample, which method comprises the steps of:
a) providing a nucleic acid sample potentially comprising a nucleotide repeat;
b) providing an oligonucleotide complementary to said nucleotide repeat;
c) annealing said oligonucleotide to said nucleic acid sample;
d) ligating at least two of said oligonucleotides annealed to said nucleic acid sample to each other using a ligase enzyme;
e) converting a ligation by-product into ATP; and
f) detecting said ATP to determine whether a ligation reaction has occured,
wherein steps a)-f) are performed simultaneously or subsequently or in any combination of subsequent steps.

4. Method for analysing the number of nucleotide repeats in a nucleic acid sample, which method comprises the steps of:
a) providing a nucleic acid sample potentially comprising a nucleotide repeat;
b) providing an oligonucleotide complementary to said nucleotide repeat;
c) annealing said oligonucleotide to said nucleic acid sample;
d) ligating at least two of said oligonucleotides annealed to said nucleic acid sample to each other using a ligase enzyme;
e) converting a ligation by-product into ATP; and
f) detecting said ATP by a luciferase-based assay as a measure of whether a ligation reaction has occured,
wherein steps a)-f) are performed simultaneously or subsequently or in any combination of subsequent steps.

5. Method for microbial typing of a nucleic acid sample, which method comprises the steps of:
a) providing a nucleic acid sample comprising at least one marker for microbial typing;
b) providing an oligonucleotide that is completely or partially complementary a to region comprising a marker for microbial typing of said nucleic acid sample;
c) annealing said oligonucleotide to said nucleic acid sample;
d) ligating at least two of said oligonucleotides annealed to said nucleic acid sample to each other using a ligase enzyme; and
e) detecting a ligation by-product to determine whether a ligation reaction has occurred; and
f) comparing the ligation pattern of the sample with a reference pattern in order to determine the microbial type,
wherein steps a)-e) are performed simultaneously or subsequently or in any combination of subsequent steps.

6. Method according to claim 1 wherein an oligonucleotide in step b) is adapted to anneal immediately outside a repeated sequence.

7. Method according to claim 1 wherein the ligation by-product is AMP.

8. Method according to claim 1 wherein step d) is performed employing a NAD+-dependent DNA-ligase.

9. Method according to claim 1 wherein step e) is performed employing a pyruvate phosphate dikinase.

10. Method according to claim 1, wherein step d) is performed employing an ATP-dependent ligase; and apyrase is added to the ligation mixture of step d) before, during or after ligation in order to reduce excess amounts of DNA ligase substrate.

11. Method according to claim 10, wherein the ATP dependent ligase is T4 DNA ligase.

12. Method according to claim 10, wherein dATP is used as a substrate for the ATP dependent ligase in step d).

13. Method according to claim 1, wherein the ligation by-product is pyrophosphate (PPi).

14. Method according to claim 1, wherein step e) is performed employing a ATP-sulfurylase.

15. Method according to claim 1, wherein the oligonucleotide employed is a mono-, di- or multimer of the repeat.

16. Method according to claim 2, wherein the oligonucleotide is complementary to, but out of phase with, said nucleotide repeat.

17. Method according to claim 16, further comprising removing unannealed oligonucleotides with an exonuclease after the detection step.

18. Method according to claim 16, further comprising inactivating unannealed oligonucleotide with a phosphatase after the detection step.

19. Method according to claim 1, wherein the nucleic acid sample is immobilised on a support.

20. Method according to claim 19, further comprising removing unannealed oligonucleotides by washing after the detection step.

21. Method according to claim 1, further comprising amplifying a nucleic acid sample prior to step a).

22. Method according to claim 4, wherein the luciferase-based assay is a luminometric assay.

23. Method according to claim 4, wherein light that is produced in a luciferase reaction is enzymatically turned off after an initial level of produced light has been reached.

24. Method according to claim 23, wherein light production is turned off by the addition of apyrase.

25. Method according to claim 1, wherein oligonucleotides complementary to a region outside a region to be analyzed are used to generate a signal by ligation or primer extension that can be used to normalize a signal obtained from a region to be analyzed.

26. Kit for performing the method according to claim 1 comprising, in separate vials, a ligase enzyme and an enzyme for converting a ligation by-product into ATP.

27. Kit according to claim 26 further comprising, in a separate vial, a luciferase enzyme.

28. Kit according to claim 26, further comprising, in a separate vial, apyrase.

29. Kit according to claim 26, further comprising oligonucleotides complementary to a nucleotide repeat, associated with a disease selected from the group consisting of: Dentatorubral pallidoluysian atrophy (DRPLA), Fragile X syndrome, Fragile site FRAXE, Huntington's disease, Kennedy's disease, Machado-Joseph disease, Myotonic dystrophy, Friedrich's ataxia, Spinocerebellar ataxia type 1, Spinocerebellar ataxia type 2, Spinocerebellar ataxia type 3, Spinocerebellar ataxia type 6, Spinocerebellar ataxia type 8 and Spinocerebellar ataxia type 12.

30. Kit according to claim 26, further comprising oligonucleotides complementary to a genetic region informative for identification of microbial species selected from the group consisting of: 16S rRNA gene, 23S rRNA gene, groEL, gyrB, rpoB, rnpB, groEL, microsatellite sequences, minisatellite sequences, VNTRs, nuclear ribosomal DNA (rDNA) array—small-subunit (SSU) (18S-like), large-subunit (LSU)(23S, 26S, or 28S-like), 5.8S rRNA genes, and internal transcribed ribosomal DNA (rDNA) spacers (ITS1 and ITS2).

31. Composition comprising a ligase enzyme and an enzyme for converting a ligation by-product into ATP.

32. Composition according to claim 31 further comprising a luciferase enzyme.

33. Composition according to claim 31 further comprising oligonucleotides complementary to a nucleotide repeat associated with a disease selected from the group consisting of: Dentatorubral pallidoluysian atrophy (DRPLA), Fragile X syndrome, Fragile site FRAXE, Huntington's disease, Kennedy's disease, Machado-Joseph disease, Myotonic dystrophy, Friedrich's ataxia, Spinocerebellar ataxia type 1, Spinocerebellar ataxia type 2, Spinocerebellar ataxia type 3, Spinocerebellar ataxia type 6, Spinocerebellar ataxia type 8 and Spinocerebellar ataxia type 12.

34. Composition according to claim 31 further comprising oligonucleotides complementary to a genetic region informative for identification of microbial species, selected from the group consisting of 16S rRNA gene, 23S rRNA gene, groEL, gyrB, rpoB, rnpB, groEL, microsatellite sequences minisatellite sequences, VNTRs, nuclear ribosomal DNA (rDNA) array—small-subunit (SSU) (18S-like), large-subunit (LSU)(23S, 26S, or 28S-like), 5.8S rRNA genes, and internal transcribed ribosomal DNA (rDNA) spacers (ITS1 and ITS2).
